(19) United States
(12) Patent Application Publication
(10) Pub. No.: US 2011/0282414 A1
(43) Pub. Date: Nov. 17, 2011

(54) SYSTEM AND METHOD FOR DEFINING NEUROSTIMULATION LEAD CONFIGURATIONS

(75) Inventors: Sridhar Kothandaraman, Valencia, CA (US); Christopher Britton Gould, Valencia, CA (US); Mun Pook Lui, Northridge, CA (US); Chester Kim, Valencia, CA (US)

(73) Assignee: BOSTON SCIENTIFIC NEUROMODULATION CORPORATION, Valencia, CA (US)

(21) Appl. No.: 13/104,826

(22) Filed: May 10, 2011

Related U.S. Application Data
(60) Provisional application No. 61/333,673, filed on May 11, 2010.

(51) Int. Cl. A61N 1/36; G06F 3/048

(52) U.S. Cl. 607/59; 715/769

(57) ABSTRACT

A method and external control device for operating a plurality of electrode leads implanted within the tissue of a patient. A virtual electrode leads in a reference lead configuration are displayed. One of the virtual electrode leads is selected. The selected virtual electrode lead is dragged, and the displace virtual electrode lead is dropped, thereby displaying the virtual electrode leads in a new lead configuration.

### RELATED APPLICATION

The present application claims the benefit under 35 U.S.C. §119 to U.S. provisional patent application Ser. No. 61/333,673, filed May 11, 2010. The foregoing application is hereby incorporated by reference into the present application in its entirety.

### FIELD OF THE INVENTION

The present invention relates to tissue stimulation systems, and more particularly, to a system and method for programming an implantable tissue stimulator.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations Peripheral Nerve Stimulation (PNS) systems have demonstrated efficacy in the treatment of chronic pain syndromes and incontinence, and a number of additional applications are currently under investigation. Also, Functional Electrical Stimulation (FES) systems such as the Freehand system by NeuroControl (Cleveland, Ohio) have been applied to restore some functionality to paralyzed extremities in spinal cord injury patients.

These implantable neurostimulation systems typically include one or more electrode carrying neurostimulation leads, which are implanted at the desired stimulation site, and a neurostimulator (e.g., an implantable pulse generator (IPG)) implanted remotely from the stimulation site, but coupled either directly to the neurostimulation lead(s) or indirectly to the neurostimulation lead(s) via a lead extension. Thus, electrical pulses can be delivered from the neurostimulator to the neurostimulation leads to stimulate the tissue and provide the desired efficacious therapy to the patient. The neurostimulation system may further comprise a handheld patient programmer in the form of a remote control (RC) to remotely instruct the neurostimulator to generate electrical stimulation pulses in accordance with selected stimulation parameters. The RC may, itself, be programmed by a clinician, for example, by using a clinician's programmer (CP), which typically includes a general purpose computer, such as a laptop, with a programming software package installed thereon.

In the context of an SCS procedure, one or more neurostimulation leads are introduced through the patient's back into the epidural space, such that the electrodes carried by the leads are arranged in a desired pattern and spacing to create an electrode array. Multi-lead configurations have been increasingly used in electrical stimulation applications (e.g., neurostimulation, cardiac resynchronization therapy, etc.). In the neurostimulation application of SCS, the use of multiple leads increases the stimulation area and penetration depth (therefore coverage), as well as enables more combinations of anodic and cathodic electrodes for stimulation, such as transverse multipolar (bipolar, tripolar, or quadra-polar) stimulation, in addition to any longitudinal single lead configuration.

Several studies have demonstrated the advantage of using narrowly spaced, parallel leads placed symmetrically on both sides of the physiological midline in improving penetration and paresthesia coverage (see J. J. Struijk and J. Holsheimer, Tripolar Spinal Cord Stimulation: Theoretical Performance of a Dual Channel System, Medical and Biological Engineering and Computing, Vol. 34, No. 4, 1996, pp. 273-279; J. Holsheimer, B. Nuttin, G. King, W. Wesselink, J. Gybels, and P. de Sutter, Clinical Evaluation of Paresthesia Steering with a New System for Spinal Cord Stimulation, Neurosurgery, Vol. 42, No. 3, 1998, pp. 541-549; Holsheimer J., Wesselink, W. A., Optimum Electrode Geometry for Spinal Cord Stimulation: the Narrow Bipole and Tripole, Medical and Biological Engineering and Computing, Vol. 35, 1997, pp. 493-497).

One type of commercially available stimulation leads is a percutaneous lead, which comprises a cylindrical body with ring electrodes, and can be introduced into contact with the affected spinal tissue through a Touhy-like needle, which passes through the skin, between the desired vertebrae, and into the epidural space above the dura layer. For unilateral pain, a percutaneous lead is placed on the corresponding lateral side of the spinal cord. For bilateral pain, a percutaneous lead is placed down the midline of the spinal cord, or two or more percutaneous leads are placed down the respective sides of the midline of the spinal cord, and if a third lead is used, down the midline of the special cord. After proper placement of the neurostimulation leads at the target area of the spinal cord, the leads are anchored in place at an exit site to prevent movement of the neurostimulation leads. To facilitate the location of the neurostimulator away from the exit point of the neurostimulation leads, lead extensions are sometimes used.

The neurostimulation leads, or the lead extensions, are then connected to the IPG, which can then be operated to generate electrical pulses that are delivered, through the electrodes, to the targeted tissue, and in particular, the dorsal column and dorsal root fibers within the spinal cord. The stimulation creates the sensation known as paresthesia, which can be characterized as an alternative sensation that replaces the pain signals sensed by the patient.

The efficacy of SCS is related to the ability to stimulate the spinal cord tissue corresponding to evoked paresthesia in the region of the body where the patient experiences pain. Thus, the working clinical paradigm is that achievement of an effective result from SCS depends on the neurostimulation lead or leads being placed in a location (both longitudinal and lateral) relative to the spinal tissue such that the electrical stimulation will induce paresthesia located in approximately the same place in the patient's body as the pain (i.e., the target of treatment). If a lead is not correctly positioned, it is possible that the patient will receive little or no benefit from an implanted SCS system. Thus, correct lead placement can mean the difference between effective and ineffective pain therapy.

As such, the CP (described briefly above) may be used to instruct the neurostimulator to apply electrical stimulation to test placement of the leads and/or electrodes inter-operatively (i.e., in the context of an operating room (OR) mapping procedure), thereby assuring that the leads and/or electrodes are implanted in effective locations within the patient. The patient may provide verbal feedback regarding the presence of paresthesia over the pain area, and based on this feedback, the lead positions may be adjusted and re-anchored if necessary. Any incisions are then closed to fully implant the system.

Post-operatively (i.e., after the surgical procedure has been completed), a fitting procedure, which may be referred to as a navigation session, may be performed using the CP to program the RC, and if applicable the IPG, with a set of stimulation parameters that best addresses the painful site, thereby optimizing or re-optimizing the therapy. Thus, the navigation session may be used to pinpoint the stimulation region or areas correlating to the pain. Such programming ability is particularly advantageous after implantation should the leads gradually or unexpectedly move, which if uncorrected, would relocate the paresthesia away from the pain site.

Whether used inter-operatively or post-operatively, a computer program, such as Bionic Navigator®, available from Boston Scientific Neuromodulation Corporation, can be incorporated in the CP to facilitate selection of the stimulation parameters. Any incisions are then closed to fully implant the system. The Bionic Navigator® is a software package that operates on a suitable PC and allows clinicians to program stimulation parameters into an external handheld programmer (referred to as a remote control). Each set of stimulation parameters, including fractionalized current distribution to the electrodes (as percentage cathodic current, percentage anodic current, or off), programmed by the Bionic Navigator® may be stored in both the Bionic Navigator® and the remote control and combined into a stimulation program that can then be used to stimulate multiple regions within the patient.

Prior to creating the stimulation programs, the Bionic Navigator® may be operated by a clinician in a “manual mode” to manually select the percentage cathodic current and percentage anodic current flowing through the electrodes based on a representation of the physical electrode arrangement displayed on the computer screen of the CP, or may be operated by the clinician in a “navigation mode” to electrically “steer” the current along the implanted leads in real-time, thereby allowing the clinician to determine the most efficacious stimulation parameter sets that can then be stored and eventually combined into stimulation programs. In the navigation mode, the Bionic Navigator® can store selected fractionalized electrode configurations that can be displayed to the clinician as marks representing corresponding stimulation regions relative to the electrode array.

The Bionic Navigator® performs current steering in accordance with one or more steering or navigation tables, which includes a series of reference electrode combinations (for a lead of 8 electrodes) with associated fractionalized current values (i.e., fractionalized electrode configurations), can be used to gradually steer electrical current from one basic electrode combination to the next, thereby electronically steering the stimulation region along the leads. The marks can then be created from selected fractionalized electrode configurations within the navigation table that can be combined with the electrical pulse parameters to create one or more stimulation programs. Further details related to the use of navigation tables are described in U.S. patent application Ser. No. 12/614,942, entitled “System and Method for Determining Appropriate Steering Tables for Distributing Stimulation Energy Among Multiple Neurostimulation Electrodes,” which is expressly incorporated herein by reference.

With respect to side-by-side electrode configurations, it is important that the CP, whether operated in a “manual mode” or “navigation mode,” have knowledge of the lead stagger (i.e., the degree to which the first electrode of one lead is vertically offset from the first electrode of another lead) either because the physician initially implanted the electrode leads in this manner to maximize the therapeutic effect of the stimulation or because the electrode leads subsequently migrated from an initially unstaggered configuration. If a representation of non-staggered leads are displayed to the user during operation of the CP in the manual mode, or if a navigation table that was designed to steer current between the electrodes of an unstaggered side-by-side lead configuration were to be used to steer current between the electrodes of a staggered side-by-side lead configuration during the navigation mode, it is possible that at least one cathode of one lead would be adjacent an anode of the other lead, thereby possibly resulting in ineffective stimulation of the patient.

There, thus, remains a need for an improved method and system for programming multiple neurostimulation leads.

### SUMMARY OF THE INVENTION

In accordance with one aspect of the present inventions, an external control device for use with a plurality of electrode leads implanted within the tissue of a patient is provided. The external control device comprises a user interface (e.g., one or more of a mouse, trackball, touchpad, and joystick or a digitizer screen) configured for receiving input from a user and displaying virtual electrode leads in a reference lead configuration. The external control device further comprises at least one processor configured for, in response to the input from the user, selecting one of the virtual electrode leads (e.g., by coupling to the one virtual electrode lead), dragging the selected virtual electrode lead (e.g., by displacing the selected virtual electrode lead relative to at least one other one of the virtual electrode leads), and dropping the displaced virtual electrode lead (e.g., lead by decoupling from the displaced virtual electrode lead), thereby displaying the virtual electrode leads in a new lead configuration.

In one embodiment, the electrode leads are implanted within the patient in a side-by-side arrangement, in which case, the reference lead configuration may be a reference lead stagger configuration, and the new lead configuration may be a new lead stagger configuration. In an optional embodiment, the user interface is configured for displaying a lead generation icon, and the processor(s) is configured for, in response to the input from the user, allowing the user to drag and drop objects from the lead generation icon to generate the virtual electrode leads in the reference lead configuration. In another embodiment, the external control device further comprises a housing containing the user interface and the processor(s).

The processor(s) may be further configured for performing a function with reference to the graphical representation of the electrode leads in the new lead configuration. In one embodiment, the function comprises programming at least one cathode and at least one anode carried by the electrode leads. In this case, the external control device can further comprise a transmitter configured for transmitting instructions to a neurostimulator to convey electrical energy between the at least one cathode and the at least one anode to create a stimulation region within tissue of the patient. In another embodiment, the virtual leads includes virtual electrodes, in which case, the function may comprise displaying stimulation parameters (e.g., fractionalized electrical current values) adjacent the virtual electrodes. In still another embodiment, the function comprises programming a control device configured for controlling electrical stimulation energy provided to the electrode leads.

In accordance with a second aspect of the present inventions, a method of operating a plurality of electrode leads implanted within the tissue (e.g., spinal cord tissue) of a patient is provided. The method comprises displaying virtual electrode leads in a reference lead configuration, selecting one of the virtual electrode leads (e.g., by coupling to the one virtual electrode lead), dragging the selected virtual electrode lead (e.g., by displacing the selected virtual electrode lead relative to at least one other one of the virtual electrode leads), and dropping the displaced virtual electrode lead, thereby displaying the virtual electrode leads in a new lead configuration (e.g., by decoupling from the displaced virtual electrode lead).

In one method, electrode leads are implanted within the patient in a side-by-side arrangement, in which case, the reference lead configuration may be a reference lead stagger configuration, and the new lead configuration may be a new lead stagger configuration. An optional method further comprises displaying a lead generation icon, and dragging and dropping objects from the lead generation icon to generate the virtual electrode leads in the reference lead configuration. Another method may further comprise performing a function with reference to displayed virtual electrode leads in the new lead configuration. In one method, the function comprises programming at least one cathode and at least one anode carried by the electrode leads. In this case, the method may further comprise conveying electrical energy between the at least one cathode and the at least one anode to create a stimulation region within tissue of the patient. In another method, the virtual electrode leads include virtual electrodes, in which case, the function may comprise displaying stimulation parameters (e.g., fractionalized electrical current values) adjacent the virtual electrodes. In still another method, the function comprises programming a control device configured for controlling electrical stimulation energy provided to the electrode leads.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is perspective view of one embodiment of a SCS system arranged in accordance with the present inventions;

FIG. 2 is a plan view of the SCS system of FIG. 1 in use with a patient;

FIG. 3 is a side view of an implantable pulse generator and a pair of stimulation leads that can be used in the SCS system of FIG. 1;

FIG. 4 is a plan view of a remote control that can be used in the SCS system of FIG. 1;

FIG. 5 is a block diagram of the internal componentry of the remote control of FIG. 4;

FIG. 6 is a block diagram of the components of a clinician programmer that can be used in the SCS system of FIG. 1;

FIG. 7 is an illustration of a lead stagger configuration screen that can be displayed by the clinician programmer of FIG. 6, wherein a graphical representation of a lead stagger is shown;

FIG. 8 is an illustration of the lead stagger configuration screen of FIG. 7, wherein a cursor for selecting one of the neurostimulation leads in the graphical representation is shown;

FIG. 9 is an illustration of the lead stagger configuration screen of FIG. 7, wherein the selected neurostimulation lead is displaced relative to the other neurostimulation lead to define a new lead stagger configuration; and

FIG. 10 an electrode programming screen, particularly showing the new graphical lead stagger configuration defined in FIG. 9.

### DETAILED DESCRIPTION OF THE EMBODIMENTS

The description that follows relates to a spinal cord stimulation (SCS) system. However, it is to be understood that the while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

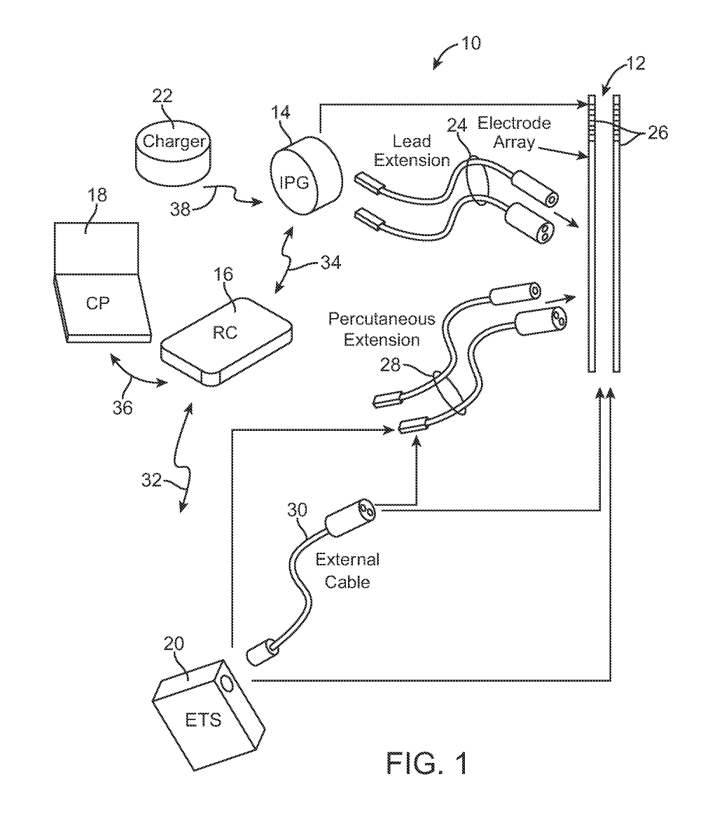

Turning first to FIG. 1, an exemplary SCS system 10 generally includes a plurality (in this case, two) of implantable neurostimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

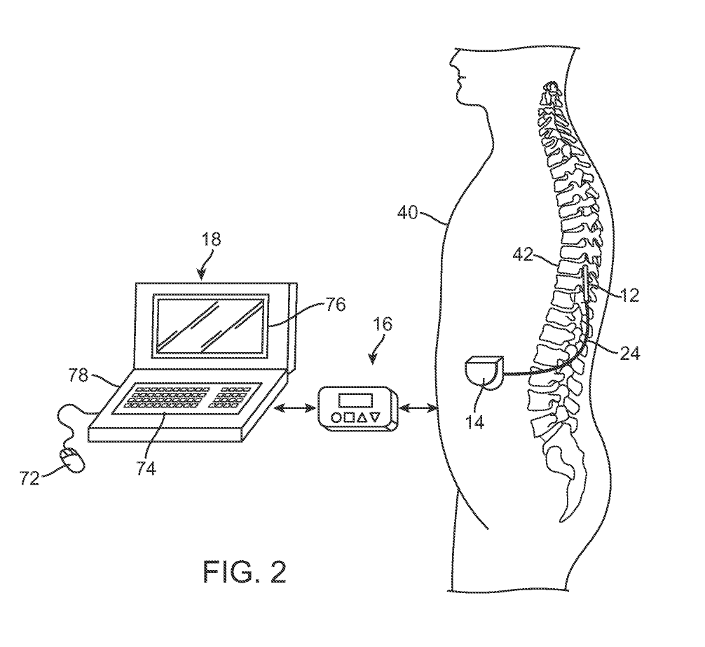

As shown in FIG. 2, the neurostimulation leads 12 are implanted within the spinal column 42 of a patient 40. The preferred placement of the neurostimulation leads 12 is adjacent, i.e., resting upon, the spinal cord area to be stimulated. Due to the lack of space near the location where the neurostimulation leads 12 exit the spinal column 42, the IPG 14 is generally implanted in a surgically-made pocket either in the abdomen or above the buttocks. The IPG 14 may, of course, also be implanted in other locations of the patient's body. The lead extension 24 facilitates locating the IPG 14 away from the exit point of the neurostimulation leads 12. As there shown, the CP 18 communicates with the IPG 14 via the RC 16.

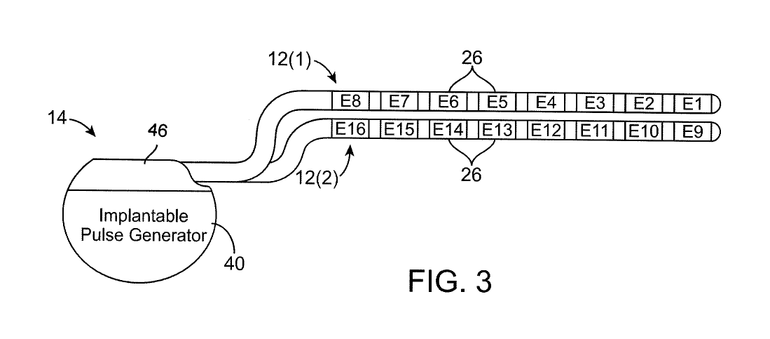

Referring now to FIG. 3, the external features of the neurostimulation leads 12 and the IPG 14 will be briefly described. One of the neurostimulation leads 12(1) has eight electrodes 26 (labeled E1-E8), and the other stimulation lead 12(2) has eight electrodes 26 (labeled E9-E16). The actual number and shape of leads and electrodes will, of course, vary according to the intended application. The IPG 14 comprises an outer case 44 for housing the electronic and other components (described in further detail below), and a connector 42 to which the proximal ends of the neurostimulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the case of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

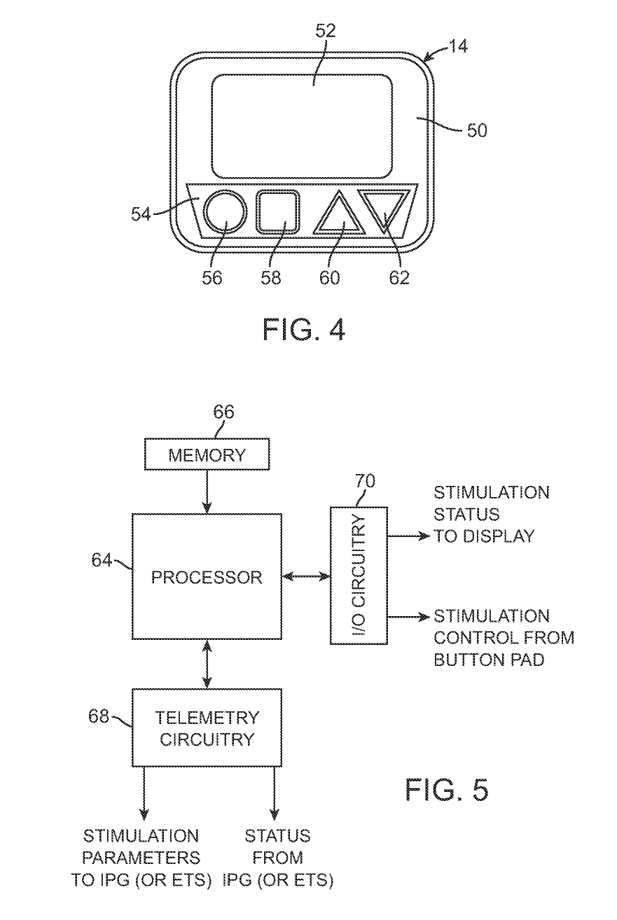

Referring now to FIG. 4, one exemplary embodiment of an RC 16 will now be described. As previously discussed, the RC 16 is capable of communicating with the IPG 14, CP 18, or ETS 20. The RC 16 comprises a casing 50, which houses internal componentry (including a printed circuit board (PCB)), and a lighted display screen 52 and button pad 54 carried by the exterior of the casing 50. In the illustrated embodiment, the display screen 52 is a lighted flat panel display screen, and the button pad 54 comprises a membrane switch with metal domes positioned over a flex circuit, and a keypad connector connected directly to a PCB. In an optional embodiment, the display screen 52 has touchscreen capabilities. The button pad 54 includes a multitude of buttons 56, 58, 60, and 62, which allow the IPG 14 to be turned ON and OFF, provide for the adjustment or setting of stimulation parameters within the IPG 14, and provide for selection between screens.

In the illustrated embodiment, the button 56 serves as an ON/OFF button that can be actuated to turn the IPG 14 ON and OFF. The button 58 serves as a select button that allows the RC 16 to switch between screen displays and/or parameters. The buttons 60 and 62 serve as up/down buttons that can be actuated to increment or decrement any of stimulation parameters of the pulse generated by the IPG 14, including pulse amplitude, pulse width, and pulse rate. For example, the selection button 58 can be actuated to place the RC 16 in a “Pulse Amplitude Adjustment Mode,” during which the pulse amplitude can be adjusted via the up/down buttons 60, 62, a “Pulse Width Adjustment Mode,” during which the pulse width can be adjusted via the up/down buttons 60, 62, and a “Pulse Rate Adjustment Mode,” during which the pulse rate can be adjusted via the up/down buttons 60, 62. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 5, the internal components of an exemplary RC 16 will now be described. The RC 16 generally includes a processor 64 (e.g., a microcontroller), memory 66 that stores an operating program for execution by the processor 64, as well as stimulation parameter sets in a navigation table (described below), input/output circuitry, and in particular, telemetry circuitry 68 for outputting stimulation parameters to the IPG 14 and receiving status information from the IPG 14, and input/output circuitry 70 for receiving stimulation control signals from the button pad 54 and transmitting status information to the display screen 52 (shown in FIG. 4). As well as controlling other functions of the RC 16, which will not be described herein for purposes of brevity, the processor 64 generates new stimulation parameter sets in response to the user operation of the button pad 54. These new stimulation parameter sets would then be transmitted to the IPG 14 (or ETS 20) via the telemetry circuitry 68. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the user (e.g., the physician or clinician) to readily determine the desired stimulation parameters to be programmed into the IPG 14, as well as the RC 16. Thus, modification of the stimulation parameters in the programmable memory of the IPG 14 after implantation is performed by a user using the CP 18, which can directly communicate with the IPG 14 or indirectly communicate with the IPG 14 via the RC 16. That is, the CP 18 can be used by the user to modify operating parameters of the electrode array 26 near the spinal cord.

As shown in FIG. 2, the overall appearance of the CP 18 is that of a laptop personal computer (PC), and in fact, may be implanted using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

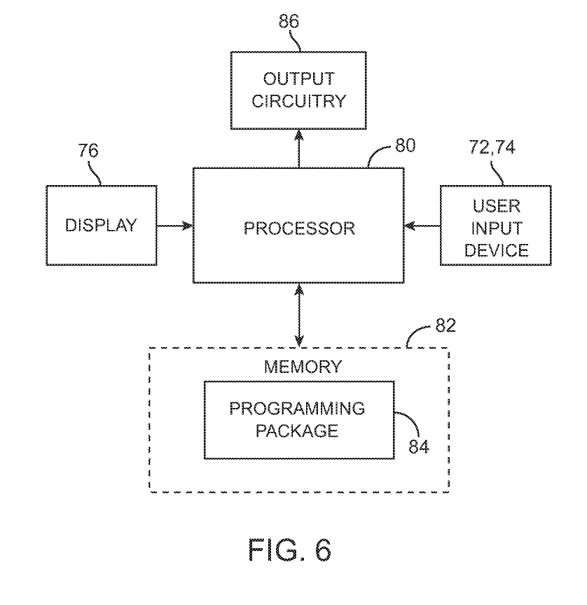

To allow the user to perform these functions, the CP 18 includes a mouse 72, a keyboard 74, and a display screen 76 housed in a case 78. In the illustrated embodiment, the display screen 76 is a conventional screen. It is to be understood that in addition to, or in lieu of, the mouse 72, other directional programming devices may be used, such as a trackball, touchpad, or joystick, can be used. Alternatively, instead of being conventional, the display screen 76 may be a digitizer screen, such as touchscreen) (not shown), may be used in conjunction with an active or passive digitizer stylus/finger touch. As shown in FIG. 6, the CP 18 generally includes a processor 80 (e.g., a central processor unit (CPU)) and memory 82 that stores a stimulation programming package 84, which can be executed by the processor 80 to allow the user to program the IPG 14, and RC 16. The CP 18 further includes output circuitry 86 (e.g., via the telemetry circuitry of the RC 16) for downloading stimulation parameters to the IPG 14 and RC 16 and for uploading stimulation parameters already stored in the memory 66 of the RC 16, via the telemetry circuitry 68 of the RC 16.

Execution of the programming package 84 by the processor 80 provides a multitude of display screens (not shown) that can be navigated through via use of the mouse 72. These display screens allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the leads 12, and select and program the IPG 14 with stimulation parameters in both a surgical setting and a clinical setting. Further details discussing the above-described CP functions are disclosed in U.S. patent application Ser. No. 12/501,282, entitled “System and Method for Converting Tissue Stimulation Programs in a Format Usable by an Electrical Current Steering Navigator,” and U.S. patent application Ser. No. 12/614,942, entitled “System and Method for Determining Appropriate Steering Tables for Distributing Stimulation Energy Among Multiple Neurostimulation Electrodes,” which are expressly incorporated herein by reference.

Most pertinent to the present inventions, programming of the IPG 14 can be performed based on a user-defined lead configuration corresponding to the actual configuration in which the leads 12 are physically implanted within the patient. In particular, the execution of the programming package 84 allows the user to define a lead configuration for storage within the CP 18. Preferably, the lead configuration is defined by the user to correspond with the actual configuration of the leads 12 within the patient, which can be obtained using suitable means, such as viewing a fluoroscopic image of the leads 12 and surrounding tissue of the patient, or using electrical means, such as transmitting electrical signals between the electrodes carried by the respective leads and measuring electrical parameters in response to the electrical signals, such as, e.g., any one or more of the manners disclosed in U.S. Pat. No. 6,993,384, entitled “Apparatus and Method for Determining the Relative Position and Orientation of Neurostimulation Leads,” U.S. patent application Ser. No. 12/550,136, entitled “Method and Apparatus for Determining Relative Positioning Between Neurostimulation Leads,” and U.S. patent application Ser. No. 12/623,976, entitled “Method and Apparatus for Determining Relative Positioning Between Neurostimulation Leads,” which are expressly incorporated herein by reference.

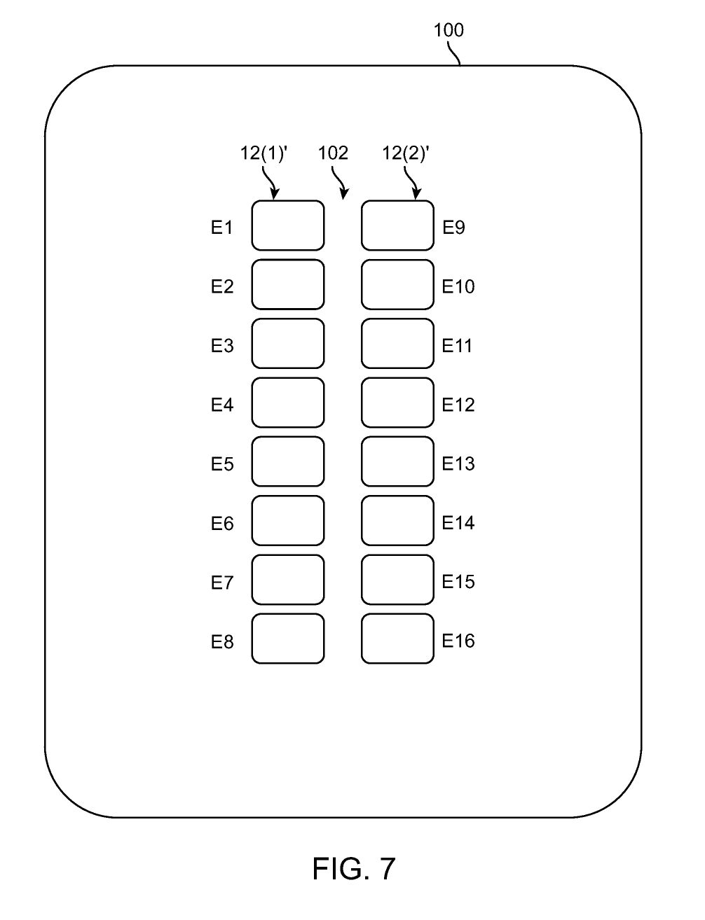

As one example, and with reference to FIG. 7, a lead configuration screen 100 illustrating a graphical representation of an adjustable lead configuration 102 (i.e., a first virtual lead 12(1)′ and second virtual lead 12(1)″) can be manipulated by the user to define the lead configuration that best matches the actual configuration of the neurostimulation leads 12. In this embodiment, the virtual leads 12′ are displayed in a side-by-side arrangement that would presumably matches the side-by-side arrangement of the actual leads 12 implanted in the patient. In other embodiments, the virtual leads 12′ may be displayed in a longitudinal arrangement (i.e., one lead completely above the other). Although the virtual leads 12′ are illustrated as being superimposed on a blank background, the virtual leads 12′ may be superimposed over a graphical representation of an anatomical region (e.g., the spine) at a location matching the location of the anatomical region at which the actual leads 12 are implanted. The lead configuration illustrated in FIG. 7 can be considered a reference lead configuration that will be adjusted to create a new lead configuration.

In one embodiment, objects can be dragged and dropped from a lead generation icon (not shown) to generate the virtual leads 12′ in a user-defined reference lead configuration. For example, one object can be dragged and dropped from the lead generation icon to generate one of the virtual leads 12(1)′, and another object can be dragged and dropped from the lead generation icon to generate another one of the virtual leads 12(2)′ relative to the previously generated virtual lead 12(1)′, such that the reference lead configuration can be user-defined. The longitudinal distance and/or lateral distance between the virtual leads 12′ can be defined in this manner.

In this embodiment, only the longitudinal distance of the virtual leads 12′ is shown as being adjusted, although in other embodiments, the lateral distance between the virtual leads 12′ may be adjusted. As shown in FIG. 7, the graphical lead configuration 102 corresponds to a configuration in which the leads 12(1) and 12(2) have no stagger. As also shown, the graphical lead configuration 102 includes the electrodes E1-E16 spaced from each other a distance equal to the spacings between the actual electrodes E1-E16. Information concerning the spacings between the electrodes E1-E16 can either be input by the user, e.g., by inputting the lead type into the CP 18, such as using the technique described in U.S. patent application Ser. No. 12/501,282, which has previously been incorporated herein by reference, or by using the electrical signaling means briefly discussed above.

The CP 18 allows the user to graphically create new lead configurations from the reference lead configuration by allowing the user to select one of the virtual leads 12′ (e.g., by coupling to one of the virtual leads 12′), dragging the selected virtual lead 12′ (e.g., by displacing the selected virtual lead 12′ relative to the other virtual lead 12′), and dropping the displaced virtual lead 12′ (e.g., by decoupling from the displaced virtual lead 12′). The manner in which a virtual lead is selected, dragged, and dropped will depend on the nature of the user interface.

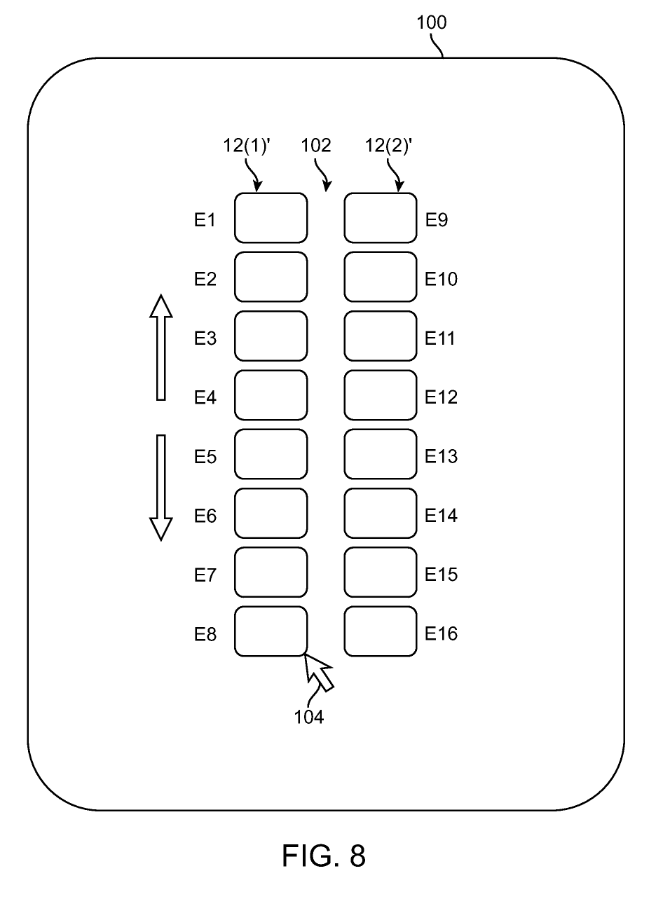

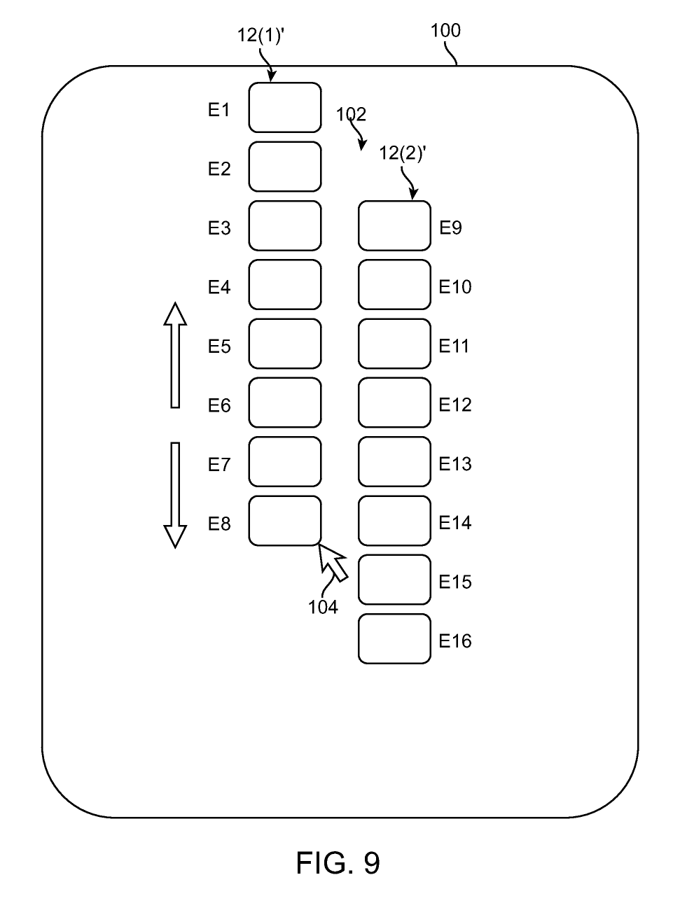

For example, if the display screen 76 is conventional, and a mouse 72 or other pointing device is used, the user may couple a cursor 104 to the first virtual lead 12(1)′ by, e.g., placing the cursor 104 adjacent to the first virtual lead 12(1)′ and clicking and holding on the appropriate button of the mouse 72, thereby selecting the first virtual lead 12(1)′, as shown in FIG. 8. The user can then move the cursor 104 to displace the first virtual lead 12(1)′ within the screen 100, thereby dragging the first virtual lead 12(1)′ relative to the second virtual lead 12(1)′, as shown in FIG. 9. For example, the cursor 104 may be moved in the direction of one of the arrows (in this case, the upward arrow) (shown in FIG. 8) to displace the first virtual lead 12(1)′ upward within the screen 100. Once the user is satisfied with the displacement of the first virtual lead 12(1)′ relative to the second virtual lead 12(2)′, the user can release the button of the mouse 72 to decouple the cursor 104 from the first virtual lead 12(1)′, thereby dropping the first virtual lead 12(1)′ to fix the configuration of the virtual leads 12′. As shown in FIG. 9, a new lead configuration has been defined by the user relative to the base lead configuration illustrated in FIG. 8, such that the first virtual lead 12(1)′ is offset upward (i.e., in the rostral direction when used during spinal cord stimulation) relative to the second virtual lead 12(2)′ by two electrodes.

As another example, if the display screen 76 is a digitizer screen, and a stylus or finger is used, the user may couple the stylus/finger to the first virtual lead 12(1)′ by, e.g., placing the stylus/finger adjacent to the first virtual lead 12(1)′ and physically touching the screen 100, thereby selecting the first virtual lead 12(1)′. The user can then move the stylus/finger across the screen 100 to displace the first virtual lead 12(1)′ within the screen 100, thereby dragging the first virtual lead 12(1)′ relative to the second virtual lead 12(1)′. Once the user is satisfied with the displacement of the first virtual lead 12(1)′ relative to the second virtual lead 12(2)′, the user can remove the stylus/finger from the screen 100 to decouple the stylus/finger from the first virtual lead 12(1)′, thereby dropping the first virtual lead 12(1)′ to fix the configuration of the virtual leads 12′.

It should be noted that although the graphical lead configuration 102 has been described as being defined by displacing the first virtual lead 12(1)′ upwards, while maintaining the second virtual lead 12(2)′ in the same position, the graphical lead configuration 102 can be similarly be defined by displacing the second virtual lead 12(2)′ downwards, while maintaining the first virtual lead 12(1)′ in the same position. It should also be noted that although the graphical lead configuration 102 has been described as being defined by displacing individual ones of the virtual leads 12′ relative to each other, the graphical lead configuration 102 can be defined by displacing a group of virtual leads relative to another group of virtual leads or an individual virtual lead. In one embodiment, lateral displacement of the virtual leads 12′ are restricted, such that the virtual leads 12′ can only be displaced in the longitudinal direction (i.e., up or down). In other embodiments, there is no restriction on displacement of the virtual leads 12′, in which case, the virtual leads 12′ may be displaced in any direction relative to each other.

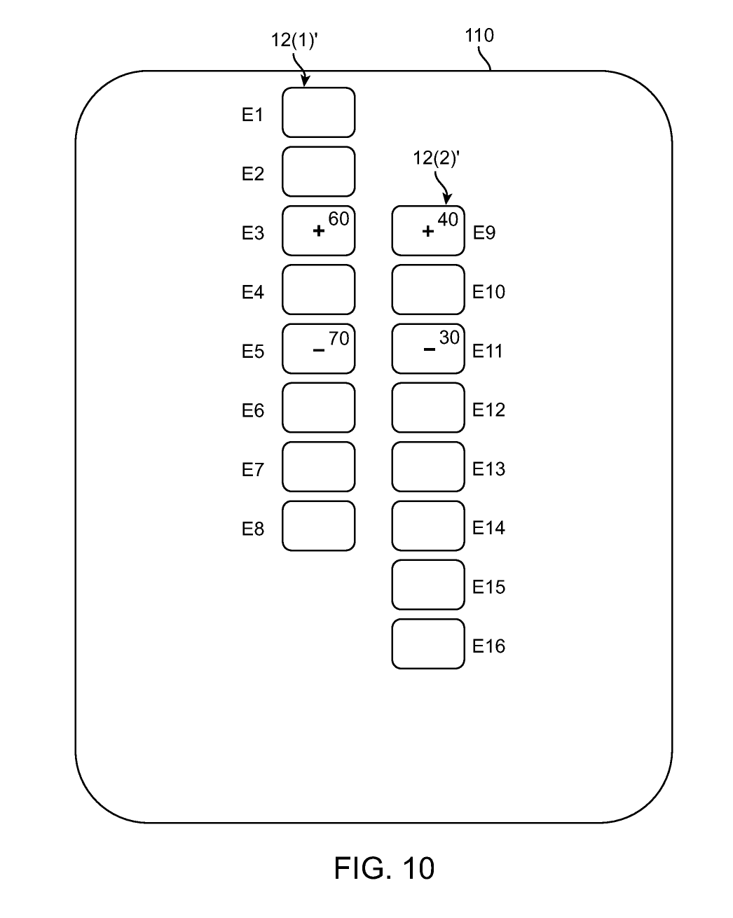

Once the lead configuration is fixed and defined (e.g., upon release of the mouse 72 button), it is then stored in the memory 82 of the CP 18 for subsequent use in programming the electrodes. For example, and with reference to FIG. 10, a programming screen 110 is shown, whereby the user may be able to program selected ones of electrodes E1-E16 as cathodes and anodes. In the illustrated embodiment, electrodes E3 and E9 are shown as being programmed as anodes (represented by plus signs), with 60% of the anodic current flowing through electrode E3 and the remaining 40% of the anodic current flowing through electrode E9, and electrodes E5 and E11 are shown as being programmed as cathodes (represented by minus signs, with 70% of the cathodic current flowing through electrode E5 and the remaining 30% of the cathodic current flowing through electrode E11). The fractionalized electrical current values are displaced adjacent, and specifically within, the graphical representations of the electrodes.

Programming of the electrodes can be performed either manually or via a current steering, as described in U.S. patent application Ser. No. 12/614,942, which has been previously incorporated herein by reference. Electrical energy can then be conveyed between the anodic electrodes E3 and E9 and the cathodic electrodes E5 and E11 to effectively create a stimulation region within the spinal cord tissue of the patient. The RC 16 may optionally be programmed for controlling the stimulation energy provided to the leads 12.

Notably, electrodes E3 and E9, which have been grouped as anodes, are immediately adjacent to each other, and electrodes E5 and E11, which have been grouped as cathodes, are immediately adjacent to each other. However, had the lead configuration not been defined by the user and a non-staggered lead configuration assumed instead, electrode E1 would have been programmed as an anode, and electrode E3 would have been programmed as a cathode, which would have resulted in ineffective stimulation of the patient, since cathodic electrode E3 would have been immediately adjacent to anodic electrode E9.

Although the foregoing lead stagger configuration technique has been described as being implemented in the CP 18, it should be noted that this technique may be alternatively or additionally implemented in the RC 16.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An external control device for use with a plurality of electrode leads implanted within the tissue of a patient, comprising:
a user interface configured for receiving input from a user and displaying virtual electrode leads in a reference lead configuration; and
at least one processor configured for, in response to the input from the user, selecting one of the virtual electrode leads, dragging the selected virtual electrode lead, and dropping the displaced virtual electrode lead, thereby displaying the virtual electrode leads in a new lead configuration.

2. The external control device of claim 1, wherein the at least one processor is configured for selecting the one virtual electrode lead by coupling to the one virtual electrode lead, configured for dragging the selected virtual electrode lead by displacing the selected virtual electrode lead relative to at least one other one of the virtual electrode leads, and configured for dropping the displaced virtual electrode lead by decoupling from the displaced virtual electrode lead.

3. The external control device of claim 1, wherein the user interface comprises one or more of a mouse, trackball, touchpad, and joystick for receiving the input from the user.

4. The external control device of claim 1, wherein the user interface comprises a digitizer screen for receiving the input from the user.

5. The external control device of claim 1, wherein the electrode leads are implanted within the patient in a side-by-side arrangement, the reference lead configuration is a reference lead stagger configuration, and the new lead configuration is a new lead stagger configuration.

6. The external control device of claim 1, wherein the user interface is configured for displaying a lead generation icon, and the at least one processor is configured for, in response to the input from the user, allowing the user to drag and drop objects from the lead generation icon to generate the virtual electrode leads in the reference lead configuration.

7. The external control device of claim 1, wherein the at least one processor is further configured for performing a function with reference to displayed virtual electrode leads in the new lead configuration.

8. The external control device of claim 7, wherein the function comprises programming at least one cathode and at least one anode carried by the electrode leads.

9. The external control device of claim 8, further comprising a transmitter configured for transmitting instructions to a neurostimulator to convey electrical energy between the at least one cathode and the at least one anode to create a stimulation region within tissue of the patient.

10. The external control device of claim 7, wherein each of the virtual electrode leads includes virtual electrodes, and the function comprises displaying stimulation parameters adjacent the virtual electrodes.

11. The external control device of claim 10, wherein the displayed stimulation parameters are fractionalized electrical current values.

12. The external control device of claim 7, wherein the function comprises programming a control device configured for controlling electrical stimulation energy provided to the electrode leads.

13. The external control device of claim 1, further comprising a housing containing the user interface and the at least one processor.

14. A method of operating a plurality of electrode leads implanted within the tissue of a patient, comprising:
displaying virtual electrode leads in a reference lead configuration;
selecting one of the virtual electrode leads;
dragging the selected virtual electrode lead; and
dropping the displaced virtual electrode lead, thereby displaying the virtual electrode leads in a new lead configuration.

15. The method of claim 14, wherein the one virtual electrode lead is selected by coupling to the one virtual electrode lead, the selected virtual electrode lead is dragged by displacing the selected virtual electrode lead relative to at least one other one of the virtual electrode leads, and the displaced virtual electrode lead is dropped by decoupling from the displaced virtual electrode lead.

16. The method of claim 14, wherein the electrode leads are implanted within the patient in a side-by-side arrangement, the reference lead configuration is a reference lead stagger configuration, and the new lead configuration is a new lead stagger configuration.

17. The method of claim 14, further comprising:
displaying a lead generation icon; and
dragging and dropping objects from the lead generation icon to generate the virtual electrode leads in the reference lead configuration.

18. The method of claim 14, further comprising performing a function with reference to displayed virtual electrode leads in the new lead configuration.

19. The method of claim 18, wherein the function comprises programming at least one cathode and at least one anode carried by the electrode leads.

20. The method of claim 19, further comprising conveying electrical energy between the at least one cathode and the at least one anode to create a stimulation region within tissue of the patient.

21. The method of claim 18, wherein each of the virtual electrode leads includes virtual electrodes, and the function comprises displaying stimulation parameters adjacent the virtual electrodes.

22. The method of claim 21, wherein the displayed stimulation parameters are fractionalized electrical current values.

23. The method of claim 18, wherein the function comprises programming a control device configured for controlling electrical stimulation energy provided to the electrode leads.

24. The method of claim 14, wherein the electrode leads are implanted adjacent the spinal cord of the patient.
